(19) United States
(12) Patent Application Publication
(10) Pub. No.: US 2009/0299467 A1
(43) Pub. Date: Dec. 3, 2009

(54) MEDICAL IMPLANT COMPRISING A BIOLOGICAL SUBSTRATE AND A DIAMOND-LIKE CARBON COATING

(76) Inventors: Ian Russell Kill, Hillingdon (GB); Joe Franks, London (GB); Anthony Walter Anson, Middlesex (GB)

Correspondence Address: Intellectual Property Dept.;Dewitt Ross & Stevens SC, 2 East Mifflin Street, Suite 600, Madison, WI, 53703-2865, US

(21) Appl. No.: 11/721,092

(22) PCT Filed: Dec. 6, 2005
(86) PCT No.: PCT/GB05/04679

§ 371 Date: Aug. 13, 2009

(30) Foreign Application Priority Data
Dec. 7, 2004 (GB) 0426841.3

(51) Int. Cl. A61F 2/06; B05D 3/06; A61F 2/04; A61F 2/08

(52) U.S. Cl. 623/1.46; 427/2.24; 623/23.71; 623/13.11

(57) ABSTRACT

Described is an implant for use in medical applications and methods of making and using the implant. The implant includes a hydrated substrate and a diamond-like carbon coating on the substrate. The substrate may be a biological substrate, such as collagen or elastin. The implant may be used to replace or restore the function of damaged tissue in a patient requiring such treatment.

The present invention relates to an implant for use in medical applications and to a method of producing such an implant.

Natural and artificial biomaterials (biocompatible materials) are now commonly used in medical applications in order to treat, augment, or replace a tissue, organ, or function of the body. Typical examples include titanium replacement hips, and Dacron™ polyester textile stents and sutures. Two major areas in which biomaterials play an important role are in soft tissue repair and in resolving occluded blood vessels.

One biomaterial used in soft tissue repair is Permacol™ (available from Tissue Science Laboratories plc). Permacol™, a biological product, is a sheet of acellular dermal collagen produced from porcine skin. During the processing of Permacol™, the material is chemically cross-linked to improve its stability when in contact with body fluids. Although Permacol™ has been used in a range of surgical techniques (Harper 2001), its use is limited by its physical properties. For example, the sheet cannot be used in situations which require an impervious barrier. In addition, movement of adjacent organs over Permacol™ is subject to mechanical wear and adhesions can form between the Permacol™ implant or graft and adjacent tissues. An adhesion is a band of scar tissue that joins together two anatomical surfaces which are separate in their normal state. Adhesions are a common problem associated with injury, surgery and grafting since binding two tissue layers together inappropriately can limit the movement of graft recipients. This is a particular problem with tendon and ligament replacements.

There are a number of techniques used to resolve the occlusion or stenosis (narrowing) of blood vessels. Typically, occluded blood vessels can be replaced by autografts or opened by inserting stents. An autograft is a graft taken from one part of the body and placed in another site on the same individual. Autografts include the greater saphenous vein, the internal mammary artery and the internal thoracic artery. Whilst these treatments are successful in the short term, around 30% of saphenous vein grafts fail within a year. The failure is usually due to recurrence of an occlusion or thrombus formation. Stents are typically tubes made of metal or plastic that are inserted into a blood vessel to keep the lumen open. Similarly, stenting has failure rates of 20 to 30% in the first three to twelve months post-stenting due to restenosis, a recurrence of the narrowing of the blood vessel.

The continuing development of medical devices including long term implants such as articular and intravascular prostheses, and short term applications such as catheters, has enhanced the effectiveness of surgical treatment. However, the introduction into a patient of a ‘foreign’ biomaterial can cause adverse reactions such as thrombus formation or inflammation. This is generally due to biochemical reactions at the interface between the implant and the patient's body.

In order to reduce adverse reactions, coatings such as heparin and phosphorylcholine (PC) may be applied to the devices in order to make them more compatible with the environment into which they are to be introduced. More recently, techniques have been developed that provide inert, wear-reducing and corrosion-reducing metal and plastic biomaterials (Long & Rack 1998; Morita et al 2004; Piconi & Maccauro 1999). Diamond-like carbon coatings, which are hard yet flexible, lubricious, chemically inert and impervious, have recently been introduced to improve the biological compatibility of implants comprising metallic and polymer substrates. For example, titanium substrates coated with DLC are anti-thrombogenic and can be used in heart valve prostheses (Jones et al. 2000). In addition, the efficacy of stenting can be increased by coating the stent with diamond-like carbon due to this anti-thrombogenic property (Alanazi et al. 2000). A recent study has shown a DLC coating on a polymethymethacrylate substrate to be biocompatible and support cell attachment in vitro (Li & Gu 2002). However, whilst coated metal and plastic implants may remain in a patient's body in the long term, the implants do not become integrated with the surrounding living tissue of the recipient.

The present invention seeks to address one or more of the above disadvantages.

According to a first aspect of the present invention, there is provided an implant for use in medical applications comprising a hydrated substrate and a diamond-like carbon coating on the substrate.

A hydrated substrate is a substrate having a matrix/scaffold-like structure and which comprises liquid. Typical examples of hydrated substrates include chitosans, gelatin, alginates, hydrogels, hydrated hyaluronic acid, glycopolymers and polypeptides.

Preferably the hydrated substrate is a biological substrate.

An advantage is that the substrate can become fully integrated into the site of placement to form a permanent bio-implant. Under optimal conditions, the implant becomes vascularised (i.e. comprising blood vessels) and cellularised (i.e comprising cells).

Preferably the biological substrate comprises protein.

The biological substrate may be derived from soft connective tissue, blood vessels, tendons or ligaments. Connective tissue is the supporting or framework tissue of the body, arising chiefly from the embryonic mesoderm. Soft connective tissue includes collagenous, elastic and reticular fibres, adipose tissue, and cartilage.

Advantageously the biological substrate comprises collagen and/or elastin.

The collagen is preferably fibrous dermal collagen. The collagen may be rat, bovine, ovine, canine, equine or porcine fibrous dermal collagen. Preferably the fibrous dermal collagen is Permacol™.

An advantage is that coating Permacol™ with DLC does not affect many inherent properties of Permacol™. Thus, the coated material can be sutured and forms a permanent soft tissue replacement that becomes integrated into the site of repair by becoming recellularised and revascularised. Moreover, coating Permacol™ with DLC endows the hybrid material with additional properties. The DLC coating is impervious and so DLC coated Permacol™ sheets can be used where it is desirable to limit exchange between two bodily compartments, for example between the bowel and peritoneum. The DLC coating is highly lubricious and thus endows the collagen substrate with a vastly reduced frictional surface. This is particularly useful for application useful in sites where there is extensive tissue-to-tissue rubbing. In addition, Permacol™ sheets coated with DLC have reduced adhesogenic properties compared with native Permacol™.

The diamond like coating may be chemically and/or physically bonded to the substrate by sputtering of a carbon target with energetic ions including argon ions by a dual ion beam or magnetron or ion enhanced deposition system, or a hydrocarbon ionizing beam source system or a plasma assisted chemical vapour deposition system or by laser ablation or by any other means of DLC deposition known in the art.

The coating may be from 0.01 to 5 μm thick, preferably from 0.1 to 2 μm thick, more preferably from 0.3 to 1 μm thick, most preferably about 0.5 μm thick In general, the thicker the coating, the more robust it is. However, too thick a coating can cause problems due to compressive stresses generated by the DLC itself.

Preferably the coating is provided on an upper surface of the substrate. The coating may be provided on an upper and a lower surface of the substrate

According to a second aspect of the present invention, there is provided a method of coating a substrate with a diamond-like carbon coating comprising the steps of:

(a) providing a substrate in hydrated form, and

(b) applying a diamond-like carbon coating to at least a part of the substrate.

The substrate is hydrated, i.e. the substrate comprises liquid, for example in interstitial spaces. An advantage of the method is that the substrate does not lose a substantial amount of fluid during coating. Thus cracks and discontinuities within the DLC coating are avoided.

Preferably the substrate is hydrated in aqueous solution. Advantageously, the substrate is hydrated by immersion in a saline, solution. The substrate may be immersed for at least one minute and preferably for two or more minutes. The substrate may be hydrated by other means, for example by pipetting saline or another liquid onto the substrate. The substrate may be hydrated with a non-volatile medium. Alternatively, the substrate may be provided hydrated in saline, and the saline substantially replaced with a non-volatile liquid prior to applying the diamond-like coating. An advantage of replacing the saline with a non-volatile liquid is that the shrinkage of the substrate during the coating process is reduced/eliminated. Accordingly, the duration of the coating step can be increased, producing a thicker coating in a single coating step. In addition, cracks in the DLC coating are prevented.

Preferably, the substrate is a biological substrate.

The coating may be applied to one or more surfaces of the substrate. The coating may be applied to an upper and a lower surface of the substrate. Where the substrate has six surfaces, the coating may be applied to four surfaces. This is particularly advantageous when the coated substrate is to be used in tendon and/or ligament repair. Whilst all the surfaces of a substrate could be coated, this is not preferred since the substrate can not become cellularised and/or vascularised.

The diamond-like carbon coating may be applied by sputtering of a carbon target with energetic ions including argon ions by a dual ion beam or magnetron or ion enhanced deposition system, or a hydrocarbon ionizing beam source system or a plasma assisted chemical vapour deposition system or by laser ablation or by any other means of DLC deposition known in the art.

However, a preferred method of applying the coating is by plasma assisted chemical vapour deposition since deposition by an ion plasma has been found to be particularly effective for the coatings and substrates contemplated, in particular since it provides an enveloping coating process instead of a directional coating process as with some of the other methods. In an enveloping coating process, the plasma surrounds the specimen. The specimen therefore can be coated without moving the specimen, regardless of whether it is flat, cylindrical etc. In a directional coating process, a cylindrical specimen would need to be rotated in a vacuum in order for the whole specimen to be covered by an ion beam.

Advantageously, the method further includes the steps of:

(a) providing at least part of the substrate to be coated in a housing containing at least one cathode,

(b) providing a plasma containing carbon ions in the housing,

(c) energizing the cathode or cathodes in the housing at a negative voltage potential and controlling the voltage potential of the cathode so as to create a diamond-like carbon coating on at least a part of the substrate.

The method may include the step of generating radio frequency ionization energy from a radio frequency device so as to ionize carbon atoms contained in the plasma in the housing. Advantageously the radio frequency device generates ionization waves of about 13.56 MHz. Preferably the radio frequency device operates at a voltage from 100 to 500 volts. Most preferably the device operates at around 300 volts in order to optimize the coating of the substrate without the substrate drying out substantially.

The method may include the step of creating a vacuum in the housing. Preferably the method includes the step of creating a vacuum at a pressure of approximately 10⁻¹ to 10⁻⁵ millibar. Most preferably, the vacuum pressure is approximately 10⁻² to 10⁻⁴ millibar to minimize loss of liquid from the substrate.

Preferably the method includes the step of introducing a carbon containing gas into the housing. Preferably the carbon containing gas is acetylene. The step of introducing the carbonaceous gas preferably raises the pressure inside the housing to about 20 to 30 millitorr.

The coating may be applied to the substrate for 10 to 600 seconds, preferably 50 to 500 seconds, more preferably 100 to 300 seconds and most preferably about 200 seconds. The coating step is kept short to maintain the liquid content of the substrate.

These deposition conditions produce a strong coating. To achieve a good coating of the biological substrate, there is a balance between pressure, voltage, temperature and rate of deposition. For a given voltage (for example 300 V), the lower the pressure the less concentrated is the plasma and accordingly, a longer time is needed to arrive at a coating of a particular thickness. Where the substrate is collagen, the important factors are the avoidance of excessive dehydration, which would lead to cracks in the coating, and avoidance of overheating, which could irreversibly affect the collagen. Our preferred range of conditions provide a reasonable thickness of coating without excessive dehydration and heating.

Advantageously, the coated substrate is at least partly rehydrated. During rehydration, at least some of the liquid lost from the tissue during the coating step is replaced. An advantage of the rehydration step is that the surface area of the coated surface is maintained. Accordingly, the coating remains intact and is less prone to cracking.

The coated substrate may be rehydrated by contacting the substrate with a non-volatile liquid or an aqueous solution. Examples of non volatile liquids include lipids, oils, dimethylsulphoxide (DMSO). Replacement with non-volatile liquids reduces/eliminates the shrinking of the substrate during subsequent coating steps.

The substrate may be rehydrated by immersion in a saline bath for at least two minutes.

The coating and rehydration steps may be repeated. Preferably the steps are repeated up to five times.

Preferred embodiments will now be described by way of example only and with reference to the Figures in which:

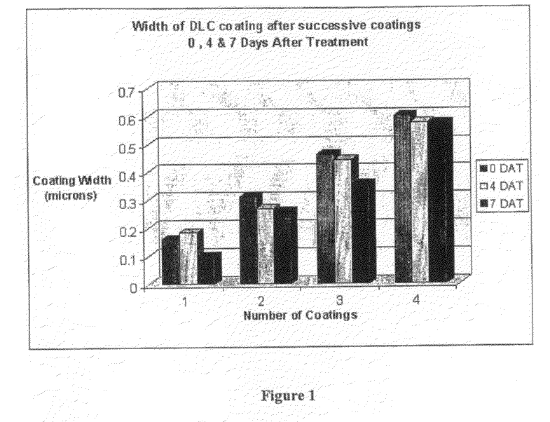

FIG. 1 is a graph showing the thickness and stability of DLC coatings on a collagenous substrate following repeated bouts of coating; and

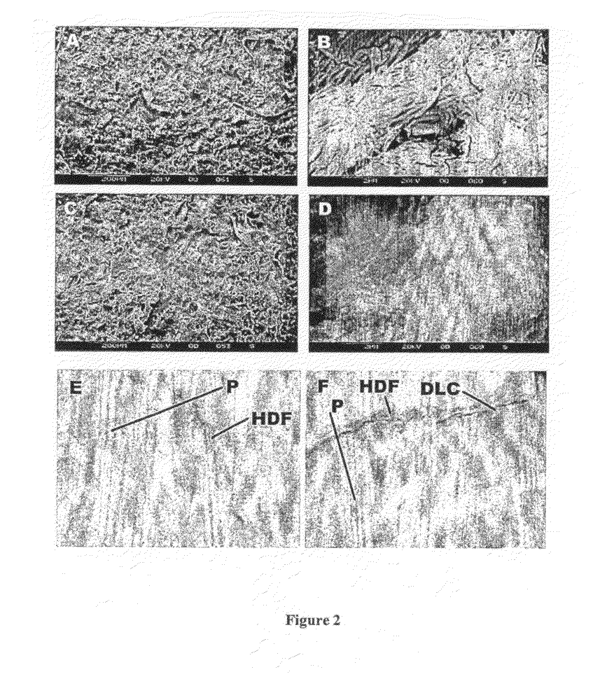

FIG. 2 comprises photographs showing that DLC coating of a collagenous substrate supports attachment of human cells and includes scanning electron microscope photographs of native Permacol™ at low power (2A) and high power (2B), and of DLC coated Permacol™ at low power (2C) and high power (2D). In its native form, Permacol™ retains the 3-dimensional structure of the dermal matrix visible under scanning electron microscopy at low power (2A). At high power, the matrix is resolved as collagen fibres (2B). Low power SEM reveals that the diamond-like carbon coating closely follows the surface contours of the matrix (2C), whilst high resolution shows that the collagen fibres are efficiently coated and no longer visible (2D). FIG. 2 further comprises photographs of Haematoxalin and Eosin staining of 8 μm wax sections prepared from native (2E) and DLC coated Permacol™ (2F) after 7 days incubation with normal human dermal fibroblasts (P=Permacol™, HDF=human dermal fibroblasts, DLC=diamond-like carbon). After 7 days the cells have formed a confluent monolayer over the surface (2E). Permacol™ coated with diamond-like carbon also supports attachment and growth of normal HDF and after 7 days in culture the cells have formed a confluent monolayer (2F).

### EXAMPLE 1

### Diamond-Like Carbon Coating of Permacol™ Sheets

Permacol™ is commercially available as sterile sheets of 5×5 cm or 5×1 cm with a thickness of either 0.75 or 1 mm. The sheets are hydrated by immersion in a saline bath and stored in saline. A film of amorphous diamond-like carbon can be layered onto one or more surfaces of the sheet using plasma assisted chemical vapour deposition (PACVD). PACVD is described in detail in GB patent no. 2,287,473, the contents of which are incorporated herein by reference. However, under normal coating conditions, the collagen becomes dehydrated and dessicated and shrinks. Upon rehydration, the collagen expands and the coating cracks. Accordingly, we have modified the procedure to allow coating of hydrated substrates.

The collagen sheet to be coated is placed on an electrode (a flat plate 250×280 mm) inside a vacuum chamber that is evacuated to a pressure about 10⁻² millibar. The number of sheets which can be coated at one time depends on the size of the electrode. The electrode is capacitively coupled to a radio frequency device. A carbon containing gas such as acetylene, which may be mixed with other gases such as argon, is introduced into the chamber, raising the pressure inside the chamber to about 20 to 30 millitorr and a plasma is formed by ionisation of the gas by the radio frequency device operating at around 300 volts. The frequency of the ionisation energy is about 13.56 MHz.

Depending on electrode geometry and RF power, the electrode will assume a negative potential for a net zero current to flow during a cycle so that current due to the positive charges carried by the low mobility ions during the greater part of the cycle is equal in magnitude to the negative current due to the electrons. The exact voltage will determine the temperature at which the coating occurs. Thus it is possible to coat at other voltages providing the temperature is not so high as to modify the substrate inappropriately.

Diamond-like carbon is produced when carbon is deposited from the plasma under energetic ion bombardment and bonds to the substrate preferentially as diamond (sp³). The instantaneous local high temperature and pressure induce a proportion of the carbon atoms to bond as diamond, the carbon being preferentially attracted to the substrate by an electrical potential. The thickness of the coating is dependent upon the time spent in the chamber under coating conditions. Initial trials revealed that prolonged periods spent in the chamber resulted in substantial loss of fluid from the collagen substrate. Although the substrate was successfully coated, upon rehydration the collagen expanded resulting in cracks and discontinuities within the DLC coating. Ideally, coating is carried out in repeated bouts of about 200 seconds. Each bout generates a coating of around 150 nm. Between each bout the material is rehydrated by placing in a saline bath for about two minutes. Under these coating conditions, the collagen does not expand substantially and so the DLC coating does not crack. The thickness of the coating can be controlled by repeated bouts of DLC application.

Referring to FIG. 1, 1 cm squares of Permacol™ were subjected to repeated bouts of coating with diamond-like carbon. Each bout of coating was for 200 seconds under around 10⁻³-10⁻⁴ millibar vacuum. Between each coating, Permacol™ squares were rehydrated in phosphate buffered saline (PBS) for 2 minutes. After coating, samples were fixed in 4% (v/v) formaldehyde in PBS for 4 hours, washed and prepared for histological examination of wax-embedded sections. Other coated samples were placed in aqueous medium (Dulbecco's Modification of Eagle's Medium) for 4 and 7 days then processed for histology as above. Sections were stained with Haematoxalin and Eosin. The width of the DLC coating after repeated bouts of coating was determined by direct measurement using a microscope equipped with an eyepiece micrometer calibrated against a stage micrometer. The durability of the coating under aqueous conditions was determined by measuring the thickness of the coating after 4 and 7 days in culture. The coating is stable in aqueous solution for at least 7 days (FIG. 1).

The duration of the coating step can be varied and depends upon the desired thickness of the coating, vacuum pressure within the coating chamber and the voltage applied through the radio frequency device. It is also possible to coat hydrated substrates at other vacuum conditions. The number of coatings is unlimited. In addition, DLC coatings can be applied to any exposed surface of the Permacol™ sheet.

Although deposition of DLC by plasma assisted chemical vapour deposition is preferred (in part because the need for manipulating the components inside the chamber during the coating process is avoided, in part because the deposition system can readily be scaled), other methods of depositing DLC can be used. Such methods included laser ablation, dual ion beam sputtering, unbalanced magnetron sputtering or other means of DLC deposition known in the art.

As shown in FIG. 2, DLC coated collagen supports attachment and growth of human cells and thus can be used as a bioimplant. Cells attach to the DLC coating but do not penetrate through the coating. However, cells, including endothelial cells which generate blood vessels, can infiltrate the collagen substrate via an uncoated surface and thus integrate the implant within the living tissue of the body.

The DLC coated substrate can be used in a range of applications. For example, DLC-coated implants reduce the extent of adhesion formation when used in hernia repair. In addition, the implant can be adapted for use within vasculature as replacement vessels or stents. Moreover, the implant can be used as a barrier where it is desirable to keep populations of cells separate.

As Permacol™ is flexible, the sheets can be distorted to form tubes and the tubular structure stabilised by suture, stapling, glueing with a biocompatible cyanoacrylate or by chemical modification of the surfaces to be joined, for example by cross-linking. Similarly, DLC coated Permacol™ can be tubularised such that the internal surface bears the DLC coating. The reduced thrombogenicity and increased lubricity of the DLC coating compared with the native Permacol™ surface makes DLC coated Permacol™ tubes ideal for use in replacing or augmenting blood vessels. The tubes can be fabricated to achieve internal diameters from 2 to 25 mm. The tube lengths will depend upon the length of the initial Permacol™ sheet used. However, the maunfacturers of Permacol™ offer a custom service to purchasers to generate desired lengths of Permacol™, at least to 30 cm.

DLC coated Permacol™ sheets can be cut to any desired shape. Coated sheets cut into strips can be used in the repair of tendon damage. Tendons connect muscle to bone and many tendon repairs fail due to formation of adhesions post injury. Permacol™ strips can be coated on all 4 exposed surfaces, excluding the end faces. The strips can then be used for example as a replacement flexor tendon by removing the damaged flexor tendon, inserting the coated Permacol™ strip into the existing tendon sheath and suturing to the stumps of the resected tendon. Alternatively, the coated Permacol™ strip can be used without a sheath and/or without connecting the tendon to the remaining tendon stumps. Other methods may be used to fix tendons to the target tissues. The same applies to ligaments which connect bone to bone or bone to cartilage. Permacol™ has good tensile and elastic properties and the coated form is lubricious and has reduced adhesiogenic properties thus reducing the risk of implant failure.

DLC coated Permacol™ sheets can be used, for example, as a repair patch in the gastrointestinal system where surgical removal of a tumour has left an aperture. The aperture can be covered with a sutured or stapled DLC coated Permacol™ patch. An advantage of this is to prevent degradation of the patch-graft due to contact with both acidic or alkali chemistry coming from the stomach or associated intestinal conduits.

### EXAMPLE 2

### Coating of Collagenous Substrates With Diamond-Like Carbon

The term fibrous dermal collagen (FDC) refers to any acellular dermal preparation of isolated mammalian pelts generated by protease treatment or freeze/thaw methods.

FDC can be prepared by acetone washing and subsequent trypsinization of rat pelts (Oliver et al 1982; Oliver et al 1975). For example, PVG/C hooded rats are sacrificed, shaved and depilated using proprietary depilation cream to remove hair over the torso. Carcasses are washed and the skin carefully removed in one large piece extending from the front to the hind legs and covering the entire torso. The underlying muscle layer and any fatty tissue present is completely removed from the inner surface. Cleaned skins are washed twice briefly using acetone (BDH, UK) and then incubated in acetone for 1 hour, 2 hr and then overnight using 100 ml fresh acetone per 5 g skin each time. This process removes any fats and lipids present within the tissue. Skins are subjected to five 1 hr washes, followed by an overnight incubation, in sterile 0.9% saline containing 0.05% (w/v) sodium azide (saline wash solution). Skins are incubated in 2 mg/ml trypsin (Sigma) in sterile PBS containing 0.05% (w/v) sodium azide (100 ml solution per 5 g skin). After 7 days the trypsin solution is decanted, the skins washed twice in sterile saline wash solution, and further incubated in fresh trypsin solution for 21 days. Collagen preparations are finally subjected to five 1 hr washes, followed by an overnight wash, in sterile saline wash solution. Throughout the procedure, all washes and incubations are carried out at 15° C. with constant agitation. FDC is stored in sterile wash solution at 4° C. until use.

Further examples include bovine fibrous dermal collagen, ovine fibrous dermal collagen, canine fibrous dermal collagen, and equine fibrous dermal collagen. The coating procedure described above in relation to Permacol™ can be applied to any collagenous substrate. Implants prepared in this way can be used in the same applications as the Permacol™ implants.

Similarly other soft connective tissues, such as tendons and ligaments, and harvested blood vessels can be rendered acellular and coated with DLC as described above. In the case of blood vessels, the coating is applied to the inner face of the vessel. This is achieved by inverting the blood vessel prior to coating then folding the blood vessel back following coating.

It is understood that the above description of the present invention is susceptible to various changes, modifications and adaptations.

### REFERENCES

Alanazi, A., Nojiri, C., Noguchi, T., Kido, T., Komatsu, Y., et al. 2000. Improved blood compatibility of DLC coated polymeric material. Asaio J 46:440-3.
Harper, C. 2001. Permacol: clinical experience with a new biomaterial. Hosp Med 62:90-5.
Jones, M. I., McColl, I. R., Grant, D. M., Parker, K. G., Parker, T. L. 2000. Protein adsorption and platelet attachment and activation, on TiN, TiC, and DLC coatings on titanium for cardiovascular applications. J Biomed Mater Res 52:413-21.
Li, D. J., Gu, H. Q. 2002. Cell attachment on diamond-like carbon coating. Bull. Mater. Sci. 25:7-13
Long, M., Rack, H. J. 1998. Titanium alloys in total joint replacement—a materials science perspective. Biomaterials 19:1621-39.
Morita, Y., Nakata, K., Kim, Y. H., Sekino, T., Niihara, K., Ikeuchi, K. 2004. Wear properties of alumina/zirconia composite ceramics for joint prostheses measured with an end-face apparatus. Biomed Mater Eng 14:263-70.
Piconi, C., Maccauro, G. 1999. Zirconia as a ceramic biomaterial. Biomaterials 20:1-25.

1. An implant for use in medical applications comprising a hydrated substrate and a diamond-like carbon coating on the substrate.

2. An implant as claimed in claim 1 wherein the hydrated substrate is a biological substrate.

3. An implant as claimed in claim 1 or 2 wherein the biological substrate comprises protein.

4. An implant as claimed in claim 2 or 3 wherein the biological substrate is derived from soft connective tissue, blood vessels, tendons and/or ligaments.

5. An implant as claimed in any of claims 2 to 4 wherein the biological substrate comprises collagen and/or elastin.

6. An implant as claimed in claim 5 wherein the collagen is fibrous dermal collagen.

7. An implant as claimed in claim 6 wherein the fibrous dermal collagen is Permacol™.

8. An implant as claimed in any preceding claim wherein the diamond like coating is chemically and/or physically bonded to the substrate by sputtering of a carbon target with energetic ions including argon ions by a dual ion beam or magnetron or ion enhanced deposition system, or a hydrocarbon ionizing beam source system or a plasma assisted chemical vapour deposition system or by laser ablation.

9. An implant as claimed in any preceding claim wherein the coating is from 0.01 to 5 μm thick.

10. An implant as claimed in any preceding claim wherein the coating is about 0.5 μm thick.

11. An implant as claimed in any preceding claim wherein the coating is provided on an upper surface of the substrate.

12. An implant as claimed in any preceding claim wherein the coating is provided on an upper and a lower surface of the substrate

13. A method of coating a substrate with a diamond-like carbon coating comprising the steps of:
(a) providing a substrate in hydrated form, and
(b) applying a diamond-like carbon coating to at least a part of the substrate.

14. A method as claimed in claim 13 wherein the substrate is a biological substrate.

15. A method as claimed in any of claims 13 or 14 wherein the substrate is hydrated in aqueous solution.

16. A method as claimed in any of claims 13 to 15 wherein the substrate is hydrated by immersion in a saline solution.

17. A method as claimed in claim 16 wherein the saline is substantially replaced with a non-volatile liquid prior to applying the diamond-like coating.

18. A method as claimed in any of claims 13 to 17 wherein the substrate has six surfaces and the coating is applied to four surfaces.

19. A method as claimed in any of claims 13 to 19 wherein the coating is applied by plasma assisted chemical vapour deposition.

20. A method as claimed in any of claims 12 to 16 further including the steps of:
(a) providing at least part of the substrate to be coated in a housing containing at least one cathode,
(b) providing a plasma containing carbon ions in the housing,
(c) energizing the cathode or cathodes in the housing at a negative voltage potential and controlling the voltage potential of the cathode so as to create a diamond-like carbon coating on at least a part of the substrate.

21. A method as claimed in claim 20 including the step of generating radio frequency ionization energy from a radio frequency device so as to ionize carbon atoms contained in the plasma in the housing.

22. A method as claimed in claim 21 wherein the radio frequency device generates ionization waves of about 13.56 MHz.

23. A method as claimed in claim 21 or 22 wherein the device is operated at around 300 volts.

24. A method as claimed in any of claims 20 to 23 including the step of creating a vacuum in the housing at a pressure of approximately 10⁻¹ to 10⁻⁵ millibar.

25. A method as claimed in any of claims 20 to 24 including the step of introducing a carbon containing gas into the housing.

26. A method as claimed in any of claims 13 to 25 wherein the coating is applied to the substrate for from 10 to 600 seconds.

27. A method as claimed in any of claims 13 to 26 wherein the coated substrate is at least partly rehydrated.

28. A method as claimed in claim 27 wherein the coated substrate is at least partly rehydrated by contacting the substrate with a non-volatile liquid.

29. A method as claimed in any of claims 13 to 28 wherein the coated substrate is configured into strips, patches, and/or tubes.

30. A method of repairing, replacing and/or restoring the function of damaged tissue in a patient including the step of attaching an implant as claimed in any of claims 1 to 12 near to or at the site of damage.

31. A method as claimed in claim 30 wherein the implant is attached to a part of the gastrointestinal system to cover an aperture in a tissue.

32. A method as claimed in claim 30 wherein the implant is attached to a tendon.

33. A method as claimed in claim 30 wherein the implant is attached to a blood vessel.
